(19) United States
(12) Patent Application Publication
(10) Pub. No.: US 2002/0165190 A1
(43) Pub. Date: Nov. 7, 2002

(54) Expression vector coding p972 gene for cancer therapy and adenovirus producing the same

(76) Inventors: Kim Dae Gun, Taejon (KR); Won-Kyung Cho, Taejon (KR); Neon-Cheol Jung, Taejon (KR); Young-Rim Seong, Taejon (KR); Dong-Soo Im, Taejon (KR); Seung-Suh Hong, Taejon (KR); Hyun-Soo Lee, Seoul (KR)

Correspondence Address: S Peter Ludwig, Darby & Darby, Post Office Box 5257, New York, NY, 10150-5257, US

(21) Appl. No.: 10/089,641

(22) PCT Filed: Jul. 20, 2001
(86) PCT No.: PCT/KR01/01295

(30) Foreign Application Priority Data
Jul. 31, 2000 (KR) 2000/44142

(51) Int. Cl. A61K 48/00; C12N 7/00; C12N 5/08; C12N 15/861

(52) U.S. Cl. 514/44; 424/93.2; 435/456; 435/235.1; 435/366; 435/320.1

(57) ABSTRACT

The present invention relates to a vector comprising P972 (also referred to as Gadd45γ, CR6 or OIG37) gene known as a gene producing a cell growth-inhibiting protein for the treatment of cancers, an recombinant adenovirus that encodes P972 gene in the cell, a method of producing the above adenovirus and a method for the treatment of cancers by using the above vector or adenovirus. The recombinant adenovirus of the present invention can be used in the treatment of various cancers including cervical cancer, breast cancerand colon cancer.

### TECHNICAL FIELD

The present invention relates to a vector comprising a cell growth-inhibiting gene for gene therapy, a recombinant adenovirus that can deliver the above gene into cells and a method of using the above recombinant adenovirus vector for cancer therapy.

### BACKGROUND ART OF THE INVENTION

Gene therapy is a technique for treating cancer or other genetic diseases, which is hard to cure by other conventional methods including those using the chemically synthesized drugs. Gene therapy uses genes, which are selected after investigating the molecular biological and biochemical cause of diseases, as a therapeutic material to produce the gene products in vivo for treatment of disease. Gene therapy has many advantages over the conventional therapy that uses chemically synthesized formulations in terms of the efficacy and the side effects, since gene therapy uses the actual gene products relating to the protection mechanism process against the disease in vivo, not the synthetically prepared drugs. In the early 1970's, scientists have begun to acknowledge the function of the genes. It has been considered that the congenital disease can be fundamentally treated by delivering many genes related to the genetic disease to patient directly. As time passes, people began to realize that the acquired diseases could also be treated by gene therapy. Since the first gene therapy by French Anderson group in the U.S.A. in September 1990 to treat a patient suffering from severe combined immunodeficiency (SCID), more than 2500 patients has been clinically treated by gene therapy up to date (Sci. Am. 263(2), 33-33B, 1990).

Anticancer therapy includes surgical operation, radiation or treatment of drug, hormone or immuno-stimulating agent. There has been a desire, however, to find a better and safer therapeutic method since the above-mentioned conventional methods have severe side effects and limited efficacy. Gene therapy that is currently tried for the treatment of cancer includes firstly the method of delivering the suicide gene such as thymidine kinase of herpes simplex virus or cytosine deaminase of E. coli into cancer cells. Nontoxic precursor molecules become cytotoxic molecules activated by the above-mentioned suicide genes. The converted cytotoxic molecules, in turn, inhibit the growth of cancer cells. Secondly, the genes that induce the immune reaction or produce cytokines can be delivered to cells. The delivered genes trigger the immune reaction that can eliminate cancer cells. Thirdly, the genes that prevent angiogenesis can be delivered into cancer cells or the cells that surround the cancer. Cancer cells, as a consequence die due to the lack of oxygen. Fourthly, as a method of using the genes that cause apoptosis of cancer cells, the tumor suppressor protein, p53 protein is usually used in the current gene therapy protocol. Recently, caspase-3, which is known to be to the programmed cell death, is beginning to be used to cause apoptosis of cancer cells. Since the effectiveness of the p53 protein as a tumor suppressor protein is widely acknowledged, various attempts have been and are being made to develop anticancer gene therapy using p53. Up to now, liposome and many different viruses have been used as gene carriers. The phase I clinical trial is being carried out by the National Cancer Institute in U.S.A. currently. The safety of gene therapy for the treatment of bladder cancer, breast cancer, lung cancer, and ovarian cancer is being tested at present, while the safety of the gene therapy for the carcinoma of the larynx and hepatoma is more or less approved.

Gene therapy is being applied in clinical cases by a variety of remedies. At an initial stage of gene therapy, the cells that have the defective gene were excised from a patient. The excised cells were transfected with the normal gene and transplanted into the patient. This method, however, has a serious shortcoming. In case the cells with the defective gene can live for a limited period of time, the treatment should be repeated periodically. A method that overcomes this problem to a certain extent is to deliver the normal gene directly into the defective cells by using the gene carriers such as viruses. This direct gene delivery method is currently investigated to apply not only the normal gene but also the suicide genes so that the suicide genes cause apoptosis of cells or make cancer cells be easily killed by other chemotherapy. The viruses that can infect the human cells are effective delivery tools of therapeutic genes and are very useful for gene therapy. More particularly, a therapeutic gene can be inserted into a viral DNA by the genetic recombination. The genetically modified virus can be mass-produced in vitro, infect the human cells directly, and then the therapeutic gene can be effectively expressed in cells. Adenovirus, especially, is very efficient in delivering genes into the human cells than other viruses that are used for gene therapy.

P972 gene is considered to function in growth arrest and/or DNA damage of cells (Takekawa M. et al., 1998. Cell 13:521-5301; Nakayama K. T. et al., 1999. J. Biol. Chem. 274:24766-24772). The cell growth is determined by the cell cycle mechanism controlled by the signals surrounding the cells or by the intracellular program. If the cell cycle is terminated, the cells go through the pathway of differentiation, apoptosis and aging. Any default of the regulation mechanism of the cell cycle results in a transition of normal cells into the cancer cells that undergo continuous divisions regardless of the extracellular signals or intracellular program. Even though the function of the P972 gene is not completely found, the cell apoptosis was reported to be induced when P972 is introduced inside the cells. Recently, it has been reported that P972 promotes the repair of the DNA damage by inducing the arrest of the cell cycle at G1 or G2. Others also reported that P972 induces the cell cycle arrest during the differentiation of the immune cells by cytokine treatment.

### SUMMARY OF THE INVENTION

The object of the present invention is to provide an expression vector that can express P972 gene inside a cell.

Another object of the present invention is to provide a recombinant adenovirus vector that can express P972 gene inside a cell.

Another object of the present invention is to provide a method of producing a recombinant adenovirus that can express P972 gene inside a cell.

### DETAILED DESCRIPTION OF THE PREFERRED EMBODIMENTS

The present invention relates to a gene carrier that can deliver P972 gene into a cell and an expression vector for gene therapy that can express P972 gene inside a cell. The expression vector of the present invention comprises P972 gene and a promoter operably linked to the same to express the P972 gene.

The P972 gene of the present invention includes, if necessary, the P972 gene modified to express the protein expressed by the wild type P972 gene or other proteins that are functionally equivalent to the same as well as the wild type P972 gene (GenBank Accession No.: AF078078). The wild type human P972 cDNA can be produced from the known P972 DNA sequence by using polymerase chain reaction (PCR).

The P972 gene of the present invention can include more than one factor that can lead to the expression of the P972 gene inside a cell. For example, the P972 gene of the present invention can include promoters, transcription response elements, enhancers, etc. As an example, CMV or SR-alpha can be selected as a promoter.

The carrier of the P972 gene in the present invention can be selected from the viral vectors including adenovirus vector, adeno-associated virus vector and retrovirus vector or non-viral vectors including liposome-mediated or ligand/poly-L-lysine conjugates.

In case the vectors are used as carriers, the type of the vectors is not limited to a certain kind. Any vector that can express the P972 gene inside a host cell can be used as a carrier.

It is preferable to use adenovirus vector as a gene carrier in the present invention.

Adenovirus is a DNA virus, whose genomic DNA is approximately 36 kbp in size. Adenovirus does not harm to human but E1 gene of adenoviral genome has transformation potential in rodent. Therefore, this region must be removed to enhance safety of the treatment.

To construct recombinant adenovirus as a gene delivery vector, E1A and E1B region are replaced with a desired gene to be replication-defective adenovirus called as first-generation adenovirus vector. And E3 region can be additionally eliminated when the insert gene is longer than 3.5 kb. Recombinant adenovirus without E1 region can be propagated in 293 cells expressing E1A and E1B proteins constitutively.

To construct recombinant adenovirus that can produce the P972 protein which is related to the growth arrest and DNA damage of cells, it is necessary to eliminate E1region from the adenovirus genomic DNA and to insert an expression cassette including the wild type P972 gene inside instead of the region.

As an example of these viruses, the present inventors have used an adenovirus expression vector pxcx2dCMV comprising an expression cassette composed of an immediately early promoter of Cytomegalovirus, a polycloning site and a polyadenyaltion signal of Simian virus 40 (SV 40). The expression vector pxcx2dCMV was obtained from Dr. Dong-Soo Im at Korea Research Institute of Bioscience and Biotechnology (52, Oun-dong, Yusong-ku, Taejon, Republic of Korea).

The present invention, therefore, provides pxcx2dCMV-p972 coding P972 gene as a vector for gene therapy.

The expression vector, pxcx2dCMV-p972 that can express P972 protein is constructed by inserting the wild-type human P972 cDNA obtained by PCR between HindIII and XhoI restriction enzyme sites of the polycloning site of the above-mentioned pxcx2dCMV expression vector (FIG. 1).

The present invention also provides a recombinant adenovirus coding P972 gene.

Also the present invention provides a method of constructing the above recombinant adenovirus that can be used in the anticancer gene therapy. The method of the present invention includes the process of screening a recombinant adenovirus that does not mixed with replication-competent adenovirus or wild type adenovirus among the recombinant adenoviruses that are obtained by co-transfection into packaging cells together with the adenovirus backbone plasmid and transfer plasmid (pxcx2dCMVP972), and the process of constructing the recombinant adenovirus AdP972 that can express P972 inside a cell. It is preferable to use pBHG10 as an adenovirus backbone plasmid. It is also preferable to use 293 cells as the packaging cells.

In the present invention, the selected recombinant adenovirus was named AdP972. The AdP972 was deposited with Korean Collection for Type Cultures (KCTC) at Korea Research Institute of Bioscience and Biotechnology (KRIBB), 52, Oun-dong, Yusong-ku, Taejon, Republic of Korea on Jun. 21, 2000 and was assigned the accession number KCTC 0806BP.

To mass-produce recombinant adenovirus vector of the present invention, the cells wherein adenovirus can be packaged is infected with the above recombinant adenovirus. More particularly, 293 cells can be used as the packaging cell line since the cell line includes genes of adenovirus E1 region in its chromosomal DNA and therefore can express E1a and E1b proteins constitutively.

It is hard to manipulate the gene of the adenovirus since the genomic DNA is as long as approximately 36 kbp. Only a partial segment of DNA, therefore, is included in the adenovirus transfer vector instead of the whole adenovirus DNA to be lead homologous recombination with adenovirus backbone plasmid inside 293 cells. The genes and proteins necessary for virus replication are provided from adenovirus backbone plasmid. In this case, the commercialized pBHG10 or pJM17 can be used as the adenoviral backbone plasmid.

As an example of the present invention, the recombinant adenovirus was constructed when pBHG10 or pJM17 and adenovirus expression vector were co-transfected into 293 cells. In general, when virus is propagated in 293 cells, approximately 1,000-10,000 adenovirus particles are produced in a single cell. The accumulated viruses in the cells can be purified by physical disruption of the host cells and by step gradient ultracentrifugation of the cell lysate.

Also the present invention provides the use of the P972 gene expression vector and/or the recombinant virus coding the P972 gene according to the present invention for the treatment of cancer. The expression vector and the recombinant adenovirus according to the present invention can be usually employed for the treatment of a variety of cancers including cervical cancer, breast cancer and colon cancer.

Particularly, the expression vector or the above virus of the present invention can infect the cancer tissue in patients, and can treat the cancer by inhibiting the progress of the cancer growth or causing the apoptosis of the cancer cells.

In the present invention, the present inventors have identified the production of P972 proteins by infecting various cancer cell lines, which do not express the P972 protein in the cells, with the recombinant adenovirus and have examined whether the cells undergo apoptosis or growth-arrest process. Particularly, MCF7 cell line, derived from the breast cancer, HeLa cell line, derived from the cervical cancer and RKO cell line, derived from the colon cancer, were used as the cancer cell lines. Also, the mouse transplanted with the human colon cancer cells was used as an animal model in the present invention. The cell lines and animal model, however, are not limited to those described herein.

In the present invention, the effect of the recombinant adenovirus in cell growth according to the present invention was quantified by measuring the viable cell number.

The method of measuring the viable cell number

After cells were seeded at 1.5×10⁵ cells in a 60 mm-diameter culture dish and cultured for 24 hours, adenovirus was treated to the cells at a concentration of 100 pfu/cell. After the cells were harvested in every 24 hours by treating them with trypsin/EDTA, the cells were mixed with 2.5% tryphan blue solution to make the final concentration of 0.07% (v/v). The number of the viable cells that were not dyed with tryphan blue was counted by using hemacytometer.

The invention will be further illustrated by the following examples. It should be understood that these examples are only intended to be illustrative and the present invention is not limited to the conditions, materials or devices described therein.

### BRIEF DESCRIPTION OF THE DRAWINGS

FIG. 1 is a genetic map of adenoviral expression vector pxcx2dCMV-P972 of the present invention.

FIG. 2 is a Western blot photograph showing the production of the P972 protein in the MCF7 and HeLa cell lines infected with recombinant adenovirus constructed by using the adenoviral expression vector of the present invention.

FIG. 3 is a photograph of the morphology of the MCF7 cell line infected with the adenovirus AdP972 of the present invention observed by using a phase contrast microscope.

FIG. 4 is a graph showing the growth inhibition of a colon cancer cell line RKO by the P972 protein produced from the adenovirus AdP972 of the present invention.

FIG. 5 is a graph showing the growth inhibition of a breast cancer cell line MCF7 by the P972 protein produced from the adenovirus AdP972 of the present invention.

FIG. 6 is a graph showing the growth inhibition effect of a cervical cancer cell line HeLa by the P972 protein produced from the adenovirus AdP972 of the present invention.

FIG. 7 is a photograph showing the anticancer effect of the adenovirus AdP972 of the present invention in an animal tumor model made by transplanting the human colon cancer cell line into nude mice.

### EXAMPLES

### Example 1

### Construction of the Adenovirus Expression Vector

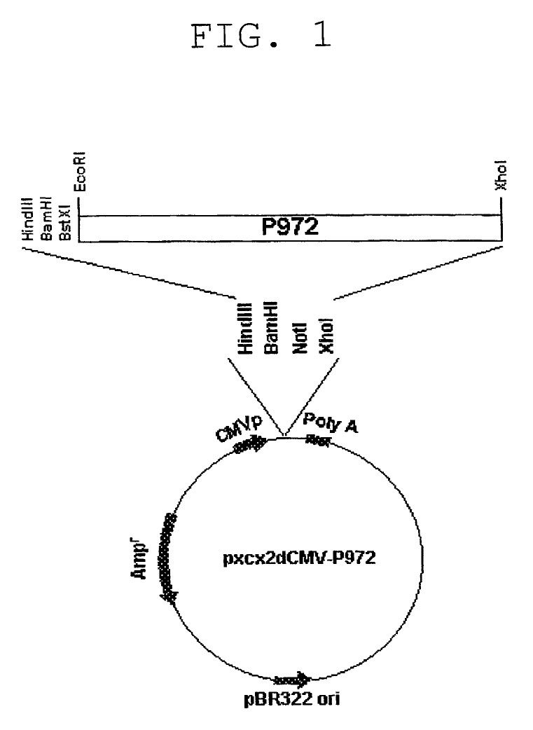

The expression vector pxcx2dCMV was used to construct the adenovirus expression vector containing the wild-type P972 gene. The wild-type P972 cDNA of 0.5 kbp in size was obtained by PCR. After digested this cDNA with the restriction enzymes HindIII and XhoI, the cDNA was inserted into pxcx2dCMV expression vector digested with the same restriction enzymes to prepare adenovirus expression vector pxcx2dCMV-P972 (FIG. 1).

### Example 2

### Preparation of the P972 antibody

After inserting the P972 gene into the vector pGEX4T (Pharmacia Inc.) that can express the P972 gene in E coli, the P972 protein was expressed in E. coli. The antibody against the P972 protein was prepared by inoculating rabbits with the purified P972 protein.

### Example 3

### Construction of the recombinant adenovirus

To construct the recombinant adenovirus that can produce P972 protein by infecting the cells, adenovirus expression vector pxcx2dCMV-P972 along with adenovirus backbone plasmid pBHG10 (Provided by Dr. Dong-Soo Im at Korea Research Institute of Bioscience and Biotechnology, Taejon, Republic of Korea) was transfected into the packaging cells, 293 cells, by the calcium phosphate method. The co-transfection was performed in a 60 mm-diameter culture dish, and the plaque formation by the virus was observed. To examine whether the constructed adenovirus AdP972 can produce P972 protein, AdP972 was infected in the breast cancer cell line, MCF7 cell line and the cervical cancer cell line, HeLa cell line and then cultured for 48 hours. Western blot analysis was performed with the antibody prepared in Example 2 to examine the expression of P972 protein in the cultured cells.

The above-obtained adenovirus was infected and proliferated in the 293 cell line cultivated in 100 mm-diameter culture dish. Then, the cells lysate in the Dulbecco's modified Eagle's medium containing 10% fetal bovine serum was performed freezing and thawing 3 times (−70° C./room temperature) to prepare adenovirus stock. The titer of the virus stock was determined by measuring the number of the plaques of the above 293 cells.

To use as a control group, adenovirus Adp53 and AdGFP coding p53 and GFP, respectively, were obtained from Dr. Dong-Soo Im in Korea Research Institute of Bioscience and Biotechnology.

### Example 4

### Western blot analysis

To confirm the expression of the P972 protein in the cell line infected with adenovirus constructed in Example 3, Western blot analysis was performed. The cells, treated with AdP972 as a concentration of 100 pfu/cell, were lysated in SDS lysis buffer solution [62.5 mM Tris-HCl (pH 6.8), 2% sodium dodecyl sulfate, 5% beta-mercaptoethanol, 10% glycerol, 0.01% bromophenol blue]. Fifty microgram of intracellular protein was separated by 14% SDS-polyacrylamide electrophoresis and transferred onto a PVDF filter paper (Millipore Co.). The above filter was blocked with phosphate buffer solution containing 0.1% Tween 20 and 5% skim milk. The protein was identified with the anti-P972 antibody prepared in the above Exmaple 2 and marked with horseradish peroxidase conjugated anti-rabbit antibody (Jackson lmmunoresearch Inc.). The protein band was visualized by observing the enhanced chemiluminescence using the ECL kit (Amersham Co., FIG. 2).

### Example 5

### Cultivation of various cell lines

The breast cancer cell line, MCF7 cell line and cervical cancer cell line, Hela cell line, were obtained from American Type Culture Collection (ATCC) and cultured in Dulbecco's modified Eagle's medium containing 10% fetal bovine serum. The colon cancer cell line, RKO cell line was obtained from A. Fornace at National Cancer Institute, USA and cultured in RPMI1640 medium containing 10% fetal bovine serum.

### Example 6

### The anticancer activity of AdP972

To investigate whether the recombinant adenovirus constructed in Example 3 can be used in anticancer gene therapy, the cancer cell lines cultured as described in Example 5 were treated with the AdP972. The effect of the expressed P972 protein in the progress of the cancer was investigated in these cell lines.

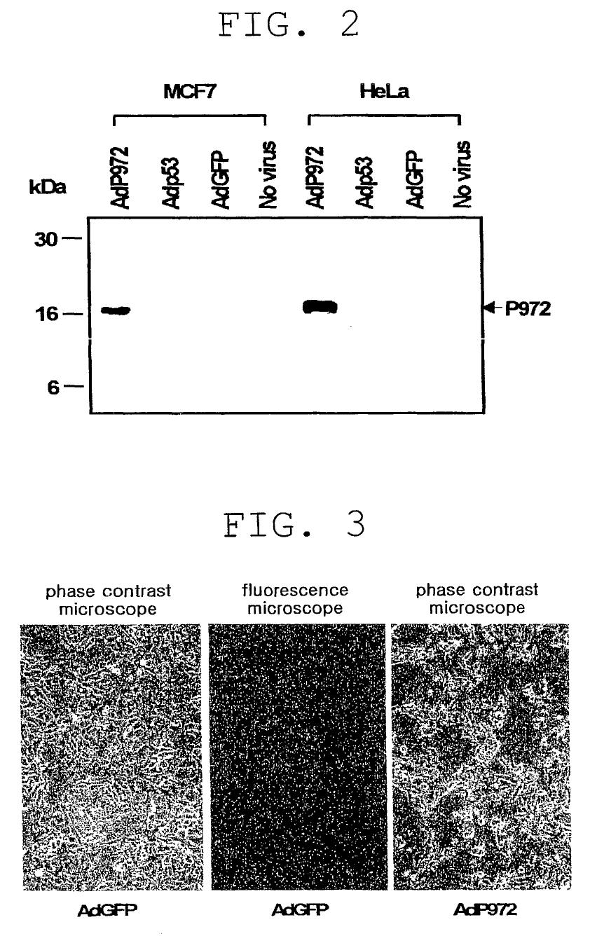

After the MCF7 cell line was treated with adenovirus AdP972 or AdGFP at a concentration of 100 pfu/cell, the cells were grown for 36 hours. The cells were observed through the phase contrast microscope and fluorescence microscope. The cell growth was greatly inhibited by the expression of P972 (FIG. 3).

The AdP972 at a concentration of 100 pfu/cell was treated to RKO cell line, MCF7 cell line and HeLa cell line to estimate the viable cell number. As a control group, the experiment was also performed in the same manner with the cells treated with the adenovirus Adp53 and AdGFP each at the concentration of 100 pfu/cell.

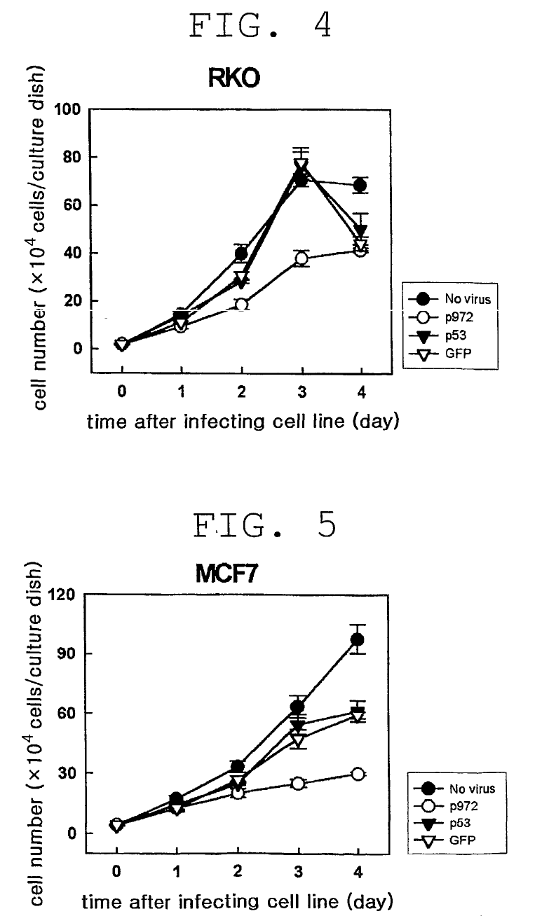

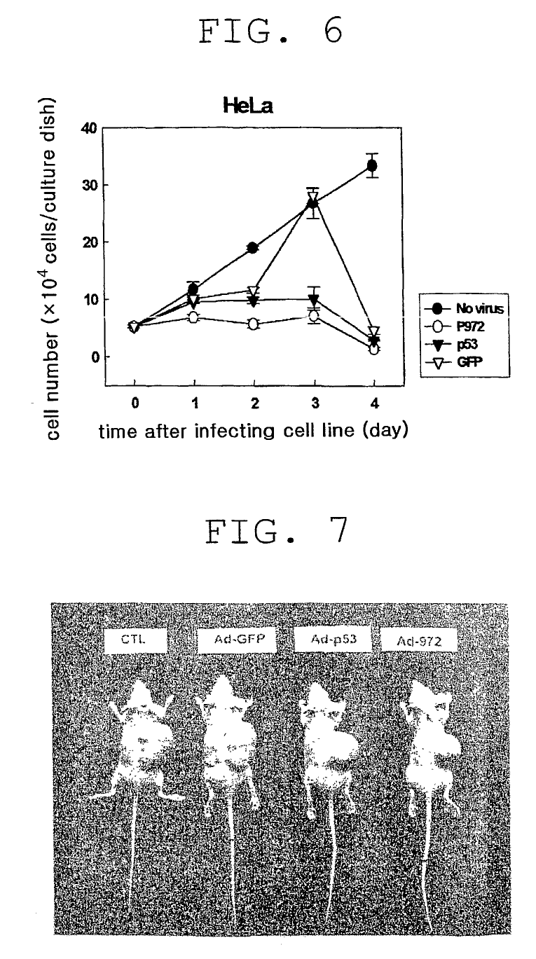

The result shows that the cell growth was greatly inhibited by the expression of P972 in the above three cancer cells and that the P972 gene has more significant effect than p53 in anticancer activity (FIGS. 4, 5 and 6).

The AdGFP adenovirus used as a control group inhibited the cell growth slightly, but the viral proteins expressed from the recombinant adenovirus, not the GFP protein, seems to be responsible for the growth retardation.

### Example 7

### Effect of AdP972 in the mouse model transplanted with the human colon cancer cells

The effect of AdP972 in the mouse model transplanted with the human colon cancer cells was examined. The HM-7 cancer cells at 1×10⁶ were injected subcutaneously on the back of the nude mouse. The cancer cells grew to 3-5 mm diameter in size after 1 week. The AdP972 was injected directly into the newly formed tumor nodule. As negative controls, PBS and AdGFP, and as a positive control, AdP53 were used. The concentration of AdGFP, AdP53 and AdP972 viruses was 1×10⁹ pfu per a mouse. To observe the tumor regression by these viruses, the tumor volume was measured for 25 days in every 5 days.

The result shows that the size of the tumor, transplanted in the nude mouse, decreased greatly by AdP972 virus. In the control group treated with AdGFP virus, however, the size of the tumor was similar to that in the group without the virus injection (FIG. 7).

### INDUSTRIAL APPLICABILITY

The expression vector including P972 of the present invention has significant effect in inducing the apoptosis and inhibiting the growth of the cancer cells. The vector of the present invention, therefore, can be used for the treatment of cancer.

1. An expression vector capable of expressing P972 comprising P972 gene and a promoter operably linked to the same to express the P972 gene.

2. The expression vector according to claim 1, wherein the said expression vector is for the treatment of cancer.

3. The expression vector according to claim 2, wherein the said cancer is breast cancer, cervical cancer or colon cancer.

4. The expression vector according to claim 1, wherein the said vector is derived from adenovirus.

5. A recombinant adenovirus containing an expression vector capable of expressing P972 comprising P972 gene and a promoter operably linked to the same to express the P972 gene.

6. A cell line transformed with an adenovirus vector of claim 5.

7. A cell line transformed with an expression vector capable of expressing P972 comprising P972 gene and a promoter operably linked to the same to express the P972 gene.

8. The cell line according to claim 7, wherein the said expression vector is for the treatment of cancer.

9. The cell line according to claim 8, wherein the said cancer is breast cancer, cervical cancer or colon cancer.

10. The cell line according to claim 7, wherein the said expression vector is an adenovirus vector.
